(19) United States
(12) Patent Application Publication
(10) Pub. No.: US 2007/0106188 A1
(43) Pub. Date: May 10, 2007

(54) Adjustable seating support system

(76) Inventor: Brock M. Walker, Okemos, MI (US)

Correspondence Address: DAVIDSON BERQUIST JACKSON & GOWDEY LLP, 4300 WILSON BLVD., 7TH FLOOR, ARLINGTON, VA, 22203, US

(21) Appl. No.: 11/588,343

(22) Filed: Oct. 27, 2006

Related U.S. Application Data
(60) Provisional application No. 60/730,855, filed on Oct. 28, 2005.

(51) Int. Cl. A61F 5/00

(52) U.S. Cl. 602/19

(57) ABSTRACT

An adjustable sacral and spinal support assembly is provided that can be used in a variety of seat types. The support assembly includes both a sacral support section and in some situations, one or more complementary spinal support sections located in other areas of the seats. The sacral support section is adapted to support the sacrum of a seated user. A method is also provided for delivering primary support to a user's sacrum and sacral-pelvic anatomy, and in select situations, secondary support to one or more of the remaining regions of the spine and/or adjacent anatomy. The support assembly is designed to produce proper spinal positioning of seated individuals to reduce fatigue, increase comfort, structural balance, stability, and posture control for a seated user. The system also adjusts and controls the load distribution from the sacral anatomy to the spine and other anatomical structures adjacent a user's sacrum, for example, the pelvis, lumbar, thoracic and cervical regions, and includes both automatic and manual adjustment systems.

### CROSS-REFERENCE TO RELATED APPLICATIONS

This application claims the benefit under 35 USC 119(e) of U.S. Provisional Application Ser. No. 60/730,855 filed Oct. 28, 2005, the entire contents of which are herein incorporated by reference.

### FIELD OF THE INVENTION

The present disclosure related generally to seating, including automotive seating, having an adjustable support system for controlling the posture and/or balance of a seated person.

### BACKGROUND OF THE INVENTION

In today's society, people spend extended amounts of time seated at work, school, home, and/or while traveling. Millions of people sit in vehicles during lengthy commutes to and from work. Once at work, they may sit continuously in an office chair for many hours without getting up. Additionally, many occupations require spending much of the day seated in an automobile. For example police officers, truck drivers and taxi cab drivers, have jobs that require spending much of the day seated in an automobile. Similarly, airplane passengers and pilots spend much of the day seated in airplane seats. There are also many individuals who must spend long periods of time in wheelchairs or other similar confining seating environments, even including musicians, where improved seating and posture control would be a benefit.

For the most part, seats have several elements in common. They have a bottom portion, or seat pan, which receives the bulk of a user's weight, and a seatback, against which the user reclines. With a focus on seatbacks, a wide variety of mechanisms have been developed that purportedly provide back support for a seat user. Most only concern support for the mid-back or lumbar region. Nevertheless millions of people continue to suffer from chronic and severe back pain caused by sitting for extended amounts of time. This is because conventional seatbacks incorrectly focus on the lumbar area and, consequently, have yet to provide overall back support that proactively resolves the seated individuals' posture, when seated, as well as the medical causes of back pain. Rather, most modern seating is only concerned with the symptoms of back pain.

By way of background, the spine has four regions: cervical (neck), thoracic (upper back), lumbar (lower back), and sacral (tail bone). The sacrum is a large triangular fusion of five vertebrae that forms the base of the spine. The sacrum is located between the pelvic bones, which include the left and right ilium. The ilia each have a posterior border portion known as the posterior superior iliac spine (“PSIS”). The lumbar region includes the five vertebrae, L₁-L₅, located above the sacrum, the thoracic region includes the twelve vertebrae, T₁-T , located above the lumbar region, and the cervical region includes the seven vertebrae, C₁-C₇, located above the thoracic region. Each region of the spine transitions into the adjacent region(s). For example, there is a thoracic-lumbar transition extending between thoracic vertebra 12 (T12) and lumbar vertebra 1 (L1), and a cervical thoracic transition between vertebras C7 and T1.

As viewed from the side, the spine of a person with good posture forms a rearward curve known as the thoracic or kyphotic curve, and two forward projecting curves known as the lumbar or lordotic curve and the cervical curve. When taken together, these three curves form an S-shaped portion of the spine. This S-Shape provides a great deal of strength, stability, flexibility, and endurance because the body primarily relies on the skeletal structures (i.e., the vertebrae) to support the weight of a persons body, rather than primarily relying on the musculature for support.

Relating to a user's posture, conventional seats have a number of shortcomings. First, conventional seats cause or permit a user's spine to collapse from an S-shaped curve into a C-shape. This collapse occurs because of improper back support. Stated differently, conventional seats lack strategically located support. Without strategically located support, the sacrum tilts rearward, and causes the spine to assume a C-shape. Use of lumbar support in a seat will not, by itself, solve or correct this posture problem. When the spine is in a C-shape, the user primarily relies on the musculature for support rather than skeletal structures. Sitting with the spine in a C-shape, and thus placing an over-reliance on the musculature for support, can lead to a number of immediate problems, for example, increased fatigue, increased pressure on the lumbar discs, or the creation of muscle stresses, strains, and spasms. Moreover, various long-term problems can also occur. These problems include pain in the lower back muscles, discomfort between the shoulder blades, tightening of neck muscles and muscle soreness and headaches.

Another problem is that conventional seats lack a contoured surface match between the surface of the seat and the surface of a user's anatomy. For example, conventional seats lack a proper nesting or receiving portion for the PSIS. In particular, the seat back pressures the PSIS. This can lead to poor posture, which often results in carrying degrees of discomfort and back or spine problems. Further, conventional seats provide poor distribution of the load forces experienced by the user.

Somewhat recently, it has been recognized that a spinal support device for applying a directed and concentrated force on the sacrum to properly position the pelvis and spine of a user could be constructed. In U.S. Pat. No. 6,125,851 (“the '851 patent”), which is commonly owned and hereby incorporated in its entirety by reference, a spinal support device is disclosed that helps support the sacrum of a user to induce the spine to take the preferable shape found in a normal standing posture.

While the '851 patent in part addresses the void created by seats around the sacral region, there still exists an urgent need to implement proper sacral support integrated within seatbacks such as those used in residential seating, office seating, and/or vehicular seating. In particular, there exists a need to provide proper sacral support in a system that is integral to a seatback and which automatically adjusts, or which can be adjusted, according to the preferences of a variety of users that differ from each other in proportion and size. Further, there exists a need to provide an efficient, responsive support system that will be useful in a wide variety of seat designs, be cost effective and yet provide improved load distribution across the surrounding pelvic area, especially around the PSIS. I have also filed U.S. patent application Ser. No. 10/900,551, filed on Jul. 27, 2004 and entitled Sacral Support Member For Seating (now Published May 19, 2005 US2005/0104428),U.S. patent application Ser. No. 11/166,341, filed on Jun. 27, 2005 and entitled Seat With Adjustable Support System, and U.S. patent application Ser. No. 11/240,617, filed on Oct. 3, 2005 and entitled Seat With Adjustable Support System, which are hereby incorporated in their entirety by reference. Each of the '851 patent, and the '341 and the '617 applications are commonly owned at the time of this filing.

A full support system is also urgently needed, including proper primary sacral support combined with secondary or complementary support for other regions of the spine.

### SUMMARY OF THE INVENTION

The present invention is directed to an improved seat support system, and more broadly to improved seating technology, especially for use in all types of vehicle seating. According to a first aspect of the present invention, the improved support apparatus delivers adjustable, specific, stabilizing support and contoured fit to a user's sacrum and sacral-pelvic anatomy, and possibly to other types of upper-back support as well. While applicable to many types of seats, this invention will work with individuals who might be wearing belts, a waist pack or belts having attachments of various kinds. The improved seat effects changes in a user's overall seated position, spinal position and overall comfort, all stemming from establishing (and maintaining) proper sacral anatomy. This results in greater and improved user efficiency, strength, and muscle control. The improved seat structure begins by actively targeting and controlling specific portions of the sacrum, thereby increasing the comfort, endurance, and stability of a user. This is accomplished by use of an adjustable mechanism, where adjustment is convenient for users who differ from each other in proportion and size. Furthermore, the improved apparatus and approach permits seats to provide anatomically engineered and controlled contoured fit for a wide variety of individuals by offering a surface match between the contours of a user's anatomy and the seatback. The improved seat can also actively target other portions of the seat user's upper-back. In addition, the structure includes improved adjustable systems, control elements and activating mechanisms that permit a wide range of adjustability yet permit convenient and easy placement within a seat.

More broadly, the present invention concerns an adjustable support system having a sacral support system and a complimentary support system, each of which is adjustable. The adjustability of the support system can control the position of an entire group of supports, of one of a group of such supports, or each of the supports, thereby providing a wide ranging level of control over the support being provided or which is available thereby. Thus, each of the user's sacral-pelvic region, thoracic-lumbar area, mid and upper thoracic area, or other parts of a user's back can be properly supported in tandem and individually. Such support ultimately can be used to control the user's overall seated posture.

In general, the sacral support system includes a sacral support member, or a sacral force transmission plate, that is configured to be moved relative to the frame which supports it, relative to a plane formed by the seat back, and relative to a seat user. This allows a user wearing belts, medical devices, or even individuals wearing a utility belt, a waist pack or other obtrusive element (even as small as a belt loop), to contact and benefit from the sacral support without creating an uncomfortable pressure zone around the user's sacral area. The movement of the sacral support member allows the seatback to comfortably accommodate or nest relative to such an obtrusive element on the back of the seated user without sacrificing seating comfort or the desired level of sacral support. The sacral support system also includes a delivery mechanism that a user can employ to adjust the amount of sacral support delivered by the sacral support system, as well as by the complementary support system for the upper portions of a seat user's back above the sacral area. The sacral support system as well as the complementary support system can be operated automatically, by using one or more sensors in the seat that monitor pressure, or manually by the seated occupant, or some combination thereof. Further, each separate device can be controlled in unison or individually. There could also be a series of preset positions in which the various systems could work in harmony, with the possibility of one or the other being turned off or rendered less or more effective that the others. In short, the present invention is contemplated as being operable in a number of ways and combinations.

As noted above, the sacral support system can be used either alone or in cooperation with a complementary support system. In general, the complementary support system includes a support member and a delivery mechanism that is also controlled as noted above. The complementary support system can be positioned within the seatback at a wide variety of positions that correspond with different areas of a user's back. For example, the complementary support system can be located within the seatback at a position corresponding with the thoracic-lumbar transition or the upper thoracic region of a user's back, or both such positions.

According to another aspect of the present invention, a novel sacral support assembly for use with a vehicle seat is provided. The sacral support assembly provides a support mechanism that primarily supports the sacrum and sacral-pelvic anatomy, but which can be paired with, or connected to, complimentary devices and structures that can provide secondary or complementary support for one or more of the remaining regions of the spine.

As used herein, the term “connected to” is intended to be interpreted broadly and to include direct and indirect connections.

As used herein, the term “vehicle” is intended to be interpreted as broadly including any transportation-related application, including, in general, for example, automobiles, trucks, racing vehicles, airplanes, boats, trains, wheelchairs, as well as household, residential, office and industrial seats and seating applications.

These and other features, aspects, and advantages of the present invention will become better understood with reference to the following description, appended claims, and accompanying drawings where:

### BRIEF DESCRIPTION OF THE DRAWINGS

FIG. 1 is a front, partial perspective view of one embodiment of the present invention;

FIG. 2 is an exploded version showing the individual elements of the embodiment of FIG. 1;

FIG. 3 is an enlarged, exploded view of the sacral support portion shown in FIG. 1;

FIG. 4 is an enlarged, exploded view of a complementary support structure shown in FIG. 1;

FIG. 5 is a side elevational view of a portion of the embodiment shown in FIG. 1, with portions having been cut away for clarity;

FIG. 6 is a diagrammatic, side elevational view showing a retracted full-line position and a dotted-line extended position;

FIG. 7 is a perspective view of a separate sacral support assembly;

FIG. 8 is a perspective view of another embodiment of the present invention;

FIG. 9 is an exploded view of the embodiment shown in FIG. 8;

FIG. 10 is a perspective view of another embodiment of the present invention;

FIG. 11 is a perspective view of the embodiment shown in FIG. 10, with portions cut away for clarity;

FIG. 12 is an exploded view of the embodiment shown in FIG. 10;

FIG. 13 is an exploded view of another embodiment of the present invention;

FIG. 14 is a front perspective view of a seat including the present invention;

FIG. 15 is a front perspective view of a seat further including load distribution members;

FIG. 16 is a front perspective view of a seat including additional support assemblies;

FIG. 17 is a front perspective view of a seat with additional load distribution material;

FIG. 18 is a rear perspective view of the use of load distribution material with a sacral support system;

FIG. 19 is a rear perspective view of the use of load distribution material with a sacral support system;

FIG. 20 is a rear perspective view of the use of load distribution material with a sacral support system;

FIG. 21 is a rear perspective view of the use of load distribution material with a sacral support system; and

FIG. 22 is a diagram of the present invention with reference to the composite structure of a seat.

### DETAILED DESCRIPTION OF THE PREFERRED EMBODIMENTS

The present invention is described with reference to the drawings in which like elements are referred to by like numerals. The relationship between and functioning of the various elements of this invention are better understood by the following detailed description. However, the embodiments of this invention as described below are by way of example only, and the invention is not limited to the embodiments illustrated in the drawings. It should also be understood that the drawings are not to scale and in certain instances details have been omitted, which are not necessary for an understanding of the present invention. Moreover, it should be noted that the invention described herein includes methodologies that have a wide variety of applications, including, those mentioned previously as well as military, and commercial seating applications.

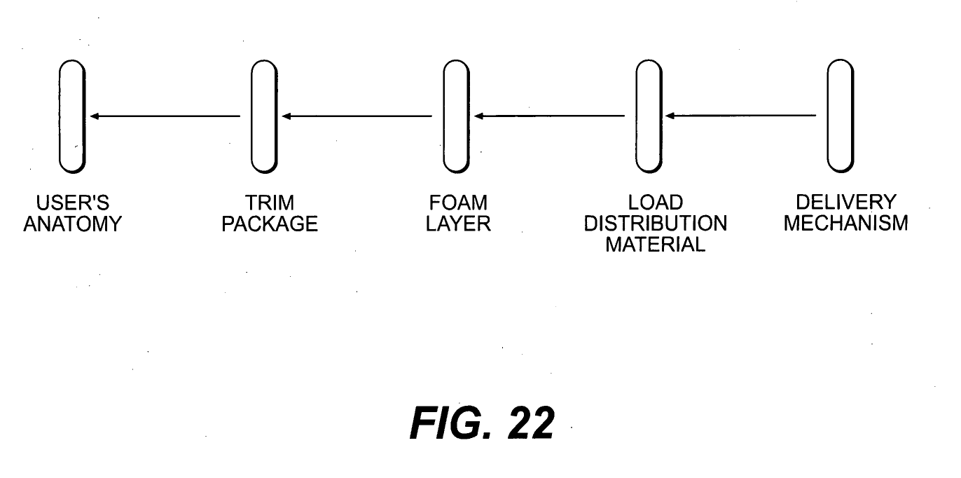

Reference can first be made to FIG. 22 which demonstrates, in a very general fashion, the concept of how the adjustable support systems, which are the subject of this invention, their individual adjustable support members, and the various delivery systems referenced hereinafter, work on a seat user's anatomy through the various layers within seating associated with seating, including vehicle seating, and, in some instances through the use of load distribution material, as defined below.

The term trim package refers to the covering used on a particular seat, such as, for example, cloth, leather or some combination of seat covering materials. The foam layer includes the various underlying layers of material used beneath the trim package including one or more foam layers, or other material used below the seat covering material. FIG. 22 shows one suggested configuration in which the delivery mechanism apples a force against a load distribution material which in turn apples a force on the foam layer, then against the trim package and finally against the seated user's anatomy. It should be understood that this represents but one possible seat configuration, and the present invention will work in this and many other types of structural seat configurations.

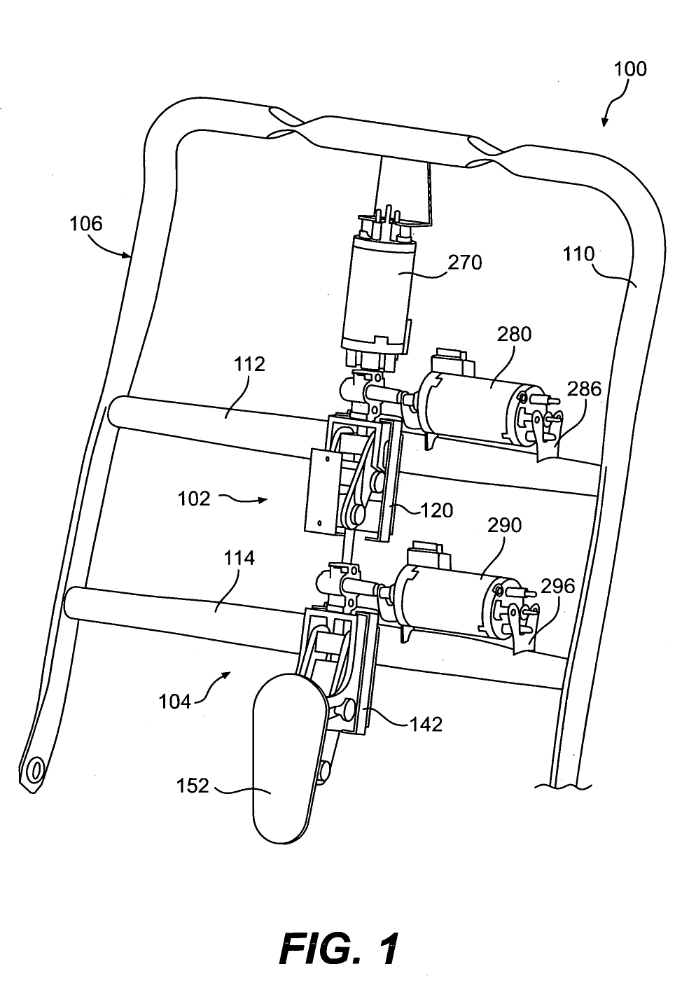

Referring to the drawings, FIGS. 1-6 illustrate a first embodiment of the present invention, and particularly, an adjustable support system having a sacral support system and a complementary support system for use with the upper back, positioned above the sacral system. The combined adjustable support system can control the position of the user's sacral-pelvic region, thoracic-lumbar area, mid and upper thoracic area, or a wide variety of other parts of a user's back. Such support ultimately can be used to control the user's overall seated posture.

In general, the combined back support system includes both an upper back support assembly and a sacral support member both of which are configured to move between retracted and extended positions along a plane formed by the seatback. It should be understood as well that either could be used separately. This allows a user, even those wearing a utility belt, a waist pack, a belt having various attachments, or other obstructive element (even as small as a belt loop), to contact either or both supports without creating an uncomfortable pressure zone around the user's lumbar and sacral areas. This allows the seatback to comfortably accommodate or nest an obstructive element, possibly being worn by the user, without sacrificing comfort of either upper-back or sacral support. The sacral and complementary support systems also include a delivery mechanism that permits a user to infinitely adjust the desired amount of sacral support delivered by the sacral support system, the amount of upper-back support and a way to correlate the forces being applied by each system to provide a comprehensive support system.

As noted above, the upper-back and sacral support systems can be used either alone or in cooperation as complementary support systems. In general, the complementary support system includes a support member and a delivery mechanism that is controlled by the user. Complementary support systems can be positioned within the seatback at a wide variety of positions that correspond with different areas of a user's back. For example, a complementary support system can be located within the seatback at a position corresponding with the thoracic-lumbar transition or the upper thoracic region if a user or both. The sacral support system and the complementary support system are each discussed in detail as follows.

Turning now to FIG. 1, the present invention comprises an adjustable support system indicated generally at 100. This system 100 is comprised of an upper-back support assembly 102, a lower or sacral support assembly 104, and a support frame 106 comprised of an outer, generally U-shaped frame 110, and two cross-members 112 and 114 that, respectively, support the upper-back support assembly 102, and the sacral support assembly 104.

It should be understood that support frame 106 is merely illustrative of a way of supporting the support assemblies within a seat. In some of the drawings only the cross members 112 or 114, or portions thereof, are shown.

As seat types vary, the internal support frame or structure can be adapted to provide a suitable connection to the sacral support assembly, and for the complementary support assembly where that system is also to be used. The present invention provides a mechanism that is easily adjusted or adapted to what ever interval support frame or device a seat or chair manufacturer might choose to use.

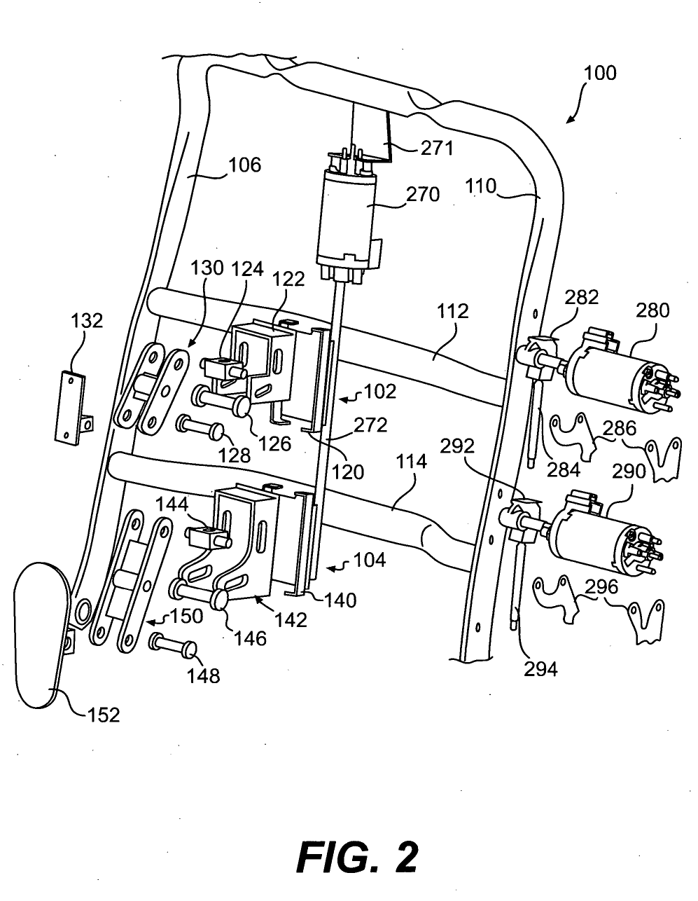

FIG. 2 shows an exploded view of the various components used to construct the support assemblies described in FIG. 1. For example, the upper-back support assembly 102 includes a rear mounting base 120, a front frame 122 in which a control side 124 is designed to move vertically. Pin 126 holds the upper part of an H-link or bracket member 130 to the lower portion of frame 122, and a support or force transmission plate 132 is pivotally supported on the H-link 130 by pin 128. The force transmission plate 132 can be about 25 mm in width and about 65 mm in height, with a thickness of about 3.35 mm.

The sacral support assembly 104 includes a rear mounting bracket 140 to which a front frame 142 is supported. A control slide 144 is designed to move and operate within the frame 142. Pin 146 holds an H-link member 150 to the lower portion of frame 142, while pin 148 holds a sacral plate 152 in a pivotal manner to the lower portion of H-link 150.

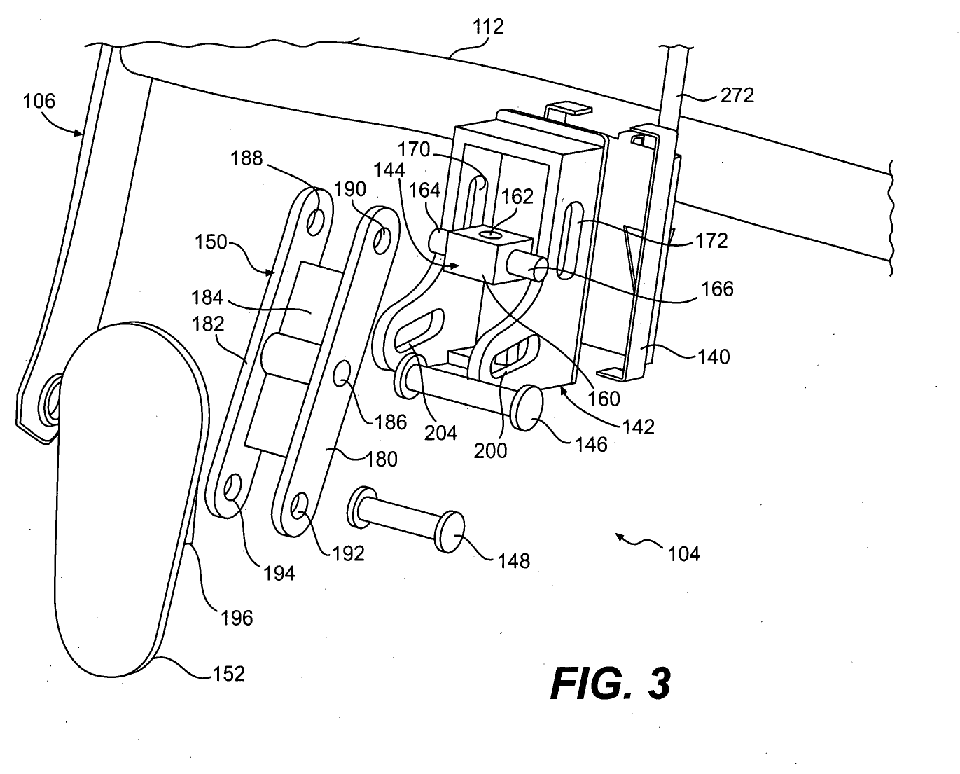

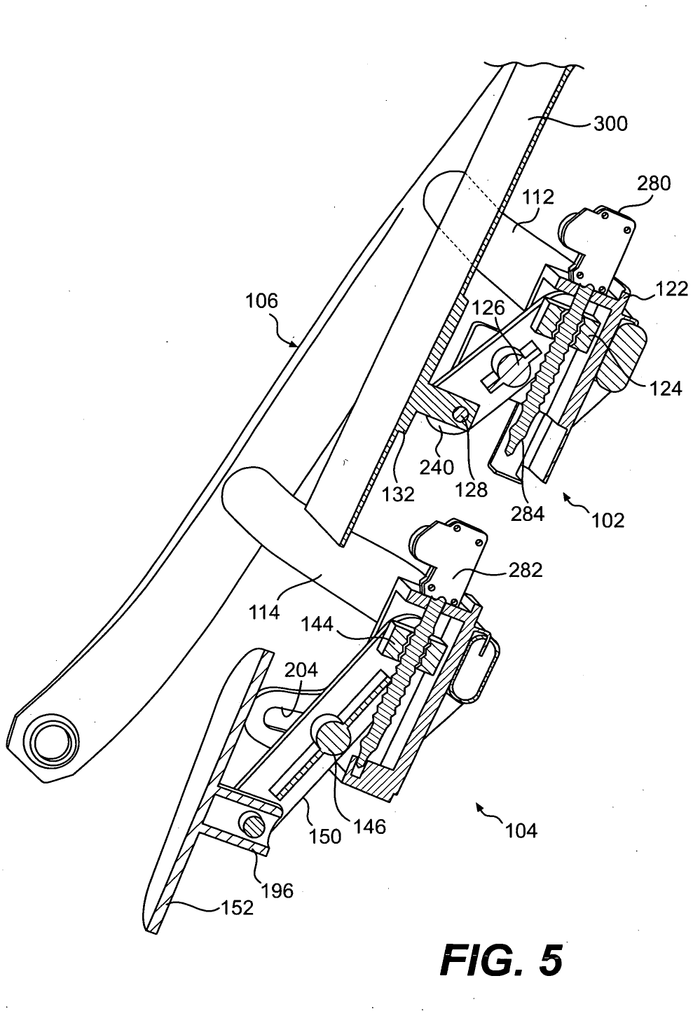

Turning now to FIG. 3, the sacral support assembly 104 includes a control slide 144 that is comprised of a central block 160 provided with a vertically extending threaded hole 162 located in the center of block 160. In addition, two cam pins, 164 and 166, extend from opposite sides of block 160 and operate, respectively, in vertically oriented cam slots 170 and 172 in the upper portion of frame 142. The H-link 150 includes two side pieces, 180 and 182, which are connected together by a central support plate 184 comprised of an enlarged central area through which a through bore or hole 186 is drilled or otherwise formed to receive pin 146. The upper portion of H-link 150 includes a pair of apertures 188 and 190, and another pair of apertures 192 and 194 are located at the bottom thereof. Apertures 192 and 194 receive the pin 148 which pivotally secures a rear portion 196, as best shown in FIG. 5, of the sacral support plate 152 to H-link 150. Apertures 188 and 190 are respectively placed over and pivotally receive the cam pins 164 and 166 of control slide 144.

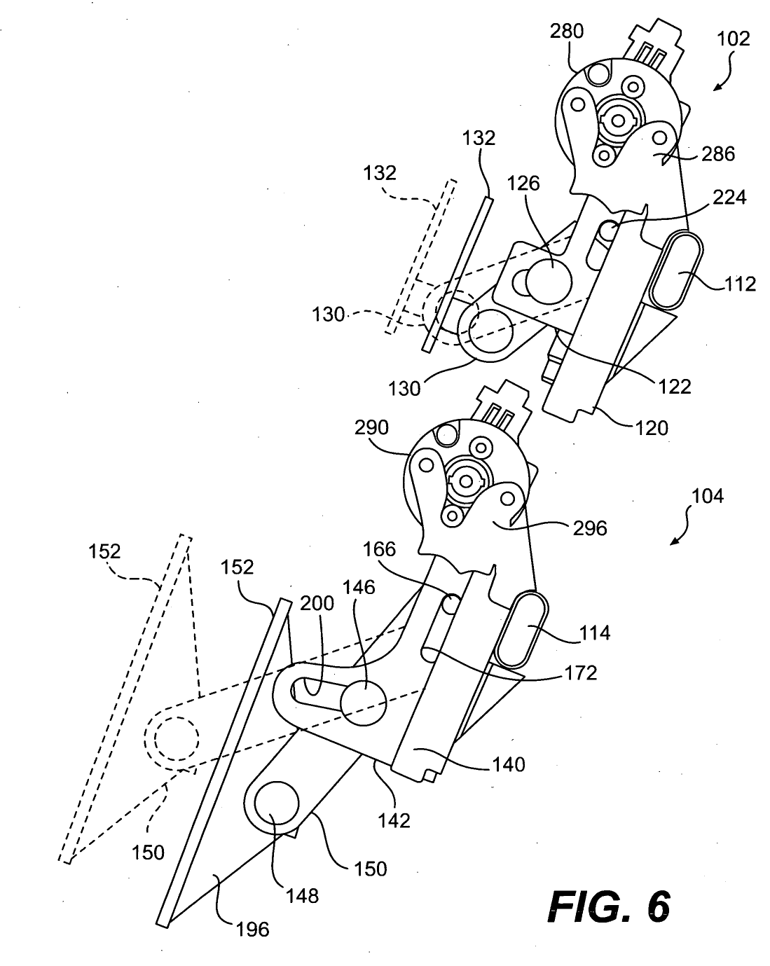

With a reference to FIG. 3, frame 142 also includes a pair of lower cam slots 200 and 204, that have an angle slightly down from horizontal, in which pin 146 can be received and permitted to slide between a rear position closest to frame 112, and a forward or extended position away from frame 112. As pin 146 slides within the cam slots 200 and 204, the central portion of H-link 150 moves back and forth which, in turn, causes the sacral plate 152 to move between retracted (rear) and extended (forward) positions. Those retracted and extended positions are shown, for example, in FIG. 6 with the retracted positions being shown in full lines and the extended positions being shown in dotted lines. The distance can vary between those retracted and extended positions, but a typical amount of total there between can be about 30 to 40 mm, and, of course, lesser amounts within the range of 0-40 mm, depending on the effect to be achieved.

Movement of the sacral support plate 152 is under the control of control slide 144. Pins 164 and 166 connect pivotally within apertures 188 and 190 as well as vertically within cam slots 170 and 172. Movement of control slide is caused by the rotation of threaded rod 294 within the threaded hole 162 as caused by motor 290 as shown in FIG. 1. Motor 290 will drive threaded rod 294 both clockwise and counterclockwise thereby driving control slide both upwardly and downwardly.

As control slide 144 moves in an upward direction, pins 164 and 166 moves the upper portion of H-link 150 upwardly in slots 170 and 172 causing pin 146 to move rearwardly and inwardly within angled cam slots 200 and 204. This moves the bottom portion of H-link 150 inwardly, and thus retracts the sacral support plate from the dotted line position toward the full line position as shown in FIG. 6. Conversely, as control slide 144 moves downwardly in slots 170 and 172, the central portion of H-link 150 moves outwardly and at an angle downwardly as pin 146 moves in slots 200 and 204. This, in turn, forces the lower part of H-link 150 outwardly and moves sacral support plate 152 from the full line position toward the extended, dotted line position shown in FIG. 6. The up and down movement of control slide 144, as controlled by motor 290, and suitable electrical controls or switching to control motor operation, is progressive, and permits an infinite number of positions for control slide 144 and, consequently, for sacral support plate 152. This allows very fine tuning and control over the position of sacral support plate 152 within its range of motion referenced above.

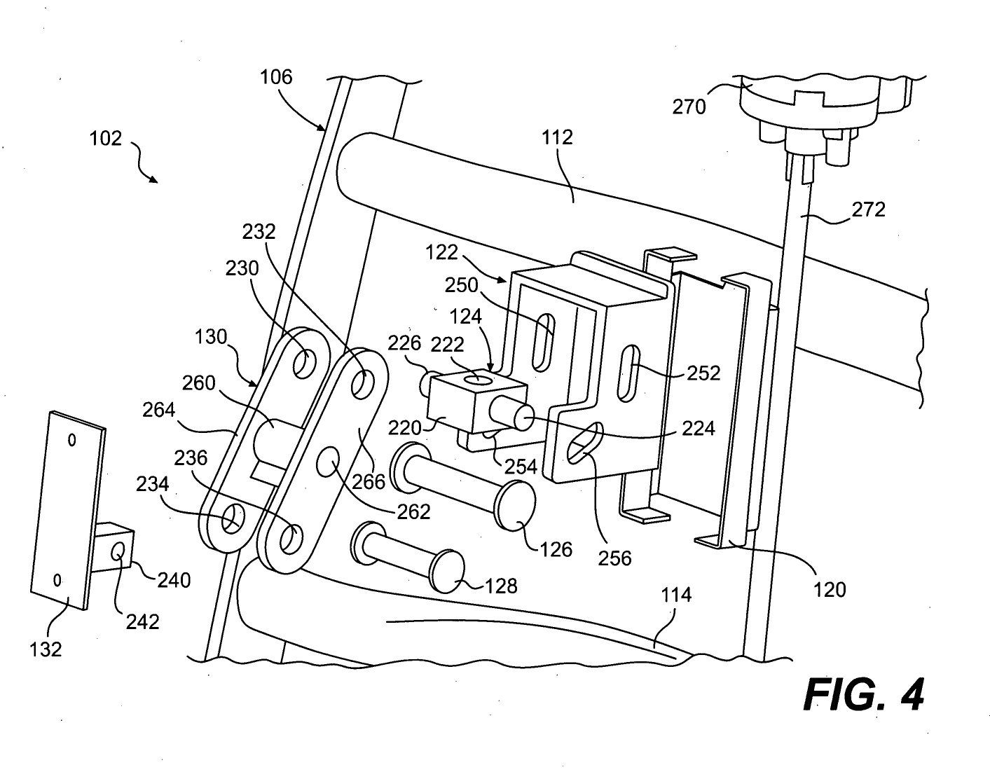

Turning to FIG. 4, the upper-back support assembly 102 includes mounting frame 122, and a control slide 124 that includes a center block 220, a threaded through bore 222, and two opposing extension pins or lugs 224 and 226 that operate, respectively, in a pair of apertures 230 and 232 in the H-link 130 and within vertical slots 250 and 252 in frame 122. H-link 130 also includes a pair of lower apertures 234 and 236 that receive the pin 128 which pivotally attaches connection member 240, that extends rearwardly from the back of plate 132 to H-link 130. Connection member 240 includes an aperture 242 through which pin 128 will pass to pivotally hold plate 132 onto H-link 130.

Frame 122 includes a pair of vertically extending cam slots, shown at 250 and 252, and a lower pair of angled cam slots 254 and 256. H-link 130 also includes a central support member 260 positioned between two side members 264 and 266. A through bore 262 is provided in support member 260, and pin 126 passes through bore 262 and through the lower angled slots 254 and 256 within frame 122 thereby holding H-link 130 to frame 122. As noted above, pins 224 and 226, on control slide 124, respectively operate in vertical cam slots 252 and 250, thereby permitting vertical movement of the control slide 124 within and relative to frame 122. Pins 224 and 226 also pivotally engage and operate within apertures 230 and 232 in H-link 130. Accordingly, movement of the control slide 124 by motor 280 and threaded rod 284 shown in FIG. 2, moves the upper portion of H-link 130 up and down depending upon the direction of rotation of rod 284. As the upper portion of H-link 130 moves down, the middle portion of H-link 130, controlled by pin 126, moves outwardly within the lower pair of angled cam slots 254 and 256 which pushes the lower portion of H-link 130 outwardly away from frame 122 toward the dotted line position in FIG. 6. As control slide 124 moves up, the force transmission plate 132 moves from the dotted line position, shown in the upper part of FIG. 6 toward the full line position. Both the up and down movement of control slide 124, and the inward and outward movement of force transmission plate are incremental and progressive, and can be controlled in an infinite number of positions, within the range of motion for plate 132 which comprises a range of about 15 to 20 mm, or 0-20 mm. This range of movement, and control thereof, permits very fine tuning and control over the position of force transmission plate 132.

The operation of both force transmission plates 132 and 152 can be controlled by either a single, or separate sets of motorized actuators each being designed to operate in two drive directions, both clockwise and counterclockwise. First, in one embodiment a single motor 270, as shown in FIG. 2, can be mounted to frame 110 by a mounting bracket 271 or other suitable mounting approach. A single threaded actuating rod 272 is drivingly connected, by a suitable coupling, to motor 290 and is engaged with and controls and moves both the sacral support plate 152 and the force transmission plate 132. Actuating rod 272 can pass through both the threaded bore 222 in control slide 124, and through the threaded bore 162 in control slide 144 so that its rotation moves both control slides 124 and 144 in unison. By actuating motor 270, the control slides 124 and 144 can be moved up and down in a coupled manner. As control slides 124 and 144 are moved up, the force transmission plates 132 and sacral support plate 152 would be retracted; conversely, as control slides 124 and 144 move downwardly, the force transmission plates 132 and sacral support plate 152 can be moved toward or into their extended dotted line positions as shown in FIG. 6.

Alternatively, each of the upper-back support assembly 102 and the sacral support assembly 104 can be independently and separately operated by motors 280 and 290, respectively. Motor 280 includes a drive assembly 282 and a depending drive rod 284 which will be threadly engaged within the threaded bore 222 of control slide 124 to operate it in the same vertical up and down fashion within frame 122. Motor 290 includes its own separate drive 292 and its depending drive rod, 294, that would operate within the thread bore 162 to control the up and down movement of control slide 144 within the cam slots 170 and 172 in frame 142. In addition, suitable support brackets 286 and 296 respectively hold motors 280 and 290 to frame elements 112 and 114 or to suitable seat frame components depending upon the seat designs.

The above embodiments have included several different operating arrangements including three motors 270, 280 and 290, along with suitable drive mechanisms and threaded drive rods, or other activating mechanisms. Each of these motors or drive systems will be able to move the system components drive to enable the raising and/or lowering of control slides 124 and 144 and the extending and retracting movement of force plates 132 and 152.

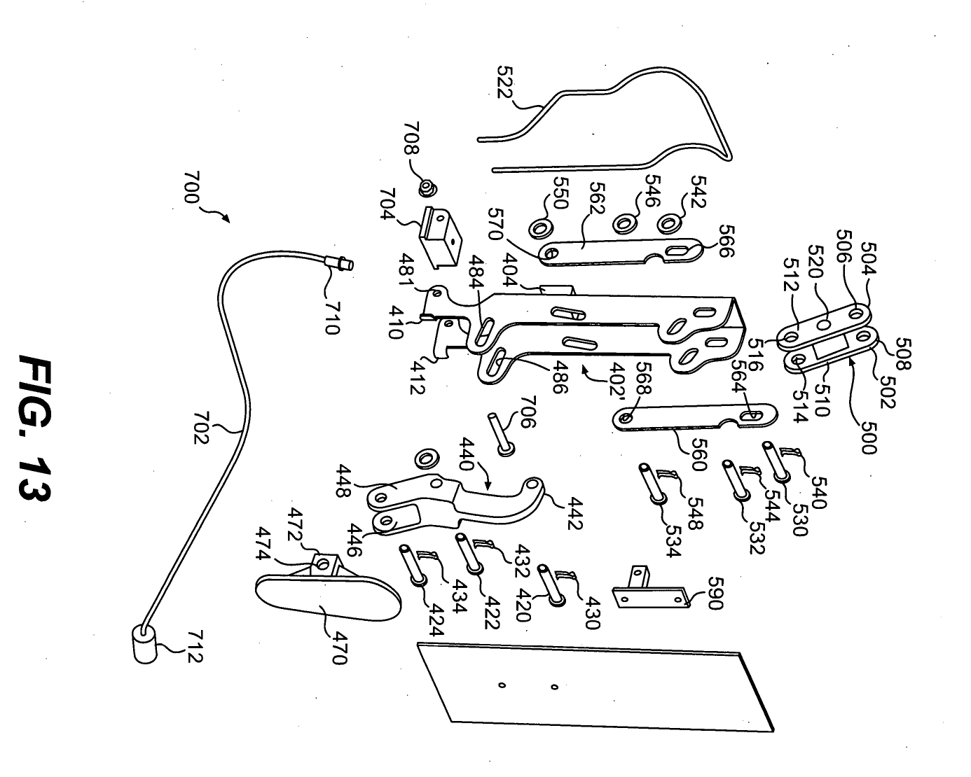

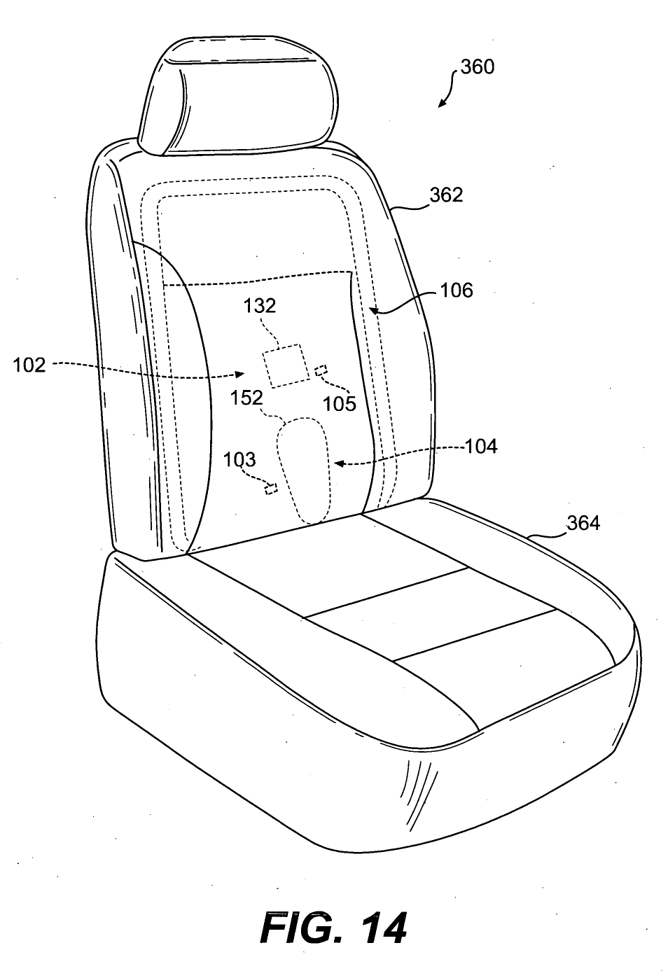

The activation of motors 270, 280 or 290, can be accomplished by separate motor control switches that could be, for example, manually operated. Alternatively, one or more, preferably a series of, sensors could be mounted in the finished seat to sense, for example, the pressures between the seat and the user, or to sense the forces being exerted and, in response to the sensed parameters, automatically move and vary the position of sacral plate 152 and/or force transmission plate 132 to provide a desired amount of pressure on the seat components and on the seated user. As is indicated above, sacral plate 152 and force transmission plate 132 can be operated together, or in tandem, or individually. Actuating control could also include a switch on the seat that could provide a selection between manual and automatic control. Further, there could be a series of preset positions of which the sacral plate 152 and/or force transmission plate 132 could be set and the switching between such preset positions could also be accomplished either manually or automatically as controlled by switch positions, by pressure or force sensors mounted in the seat cushion, such as shown at 103 and 105 in FIG. 14, respectively, for sacral plate 152 and force transmission plate 132 or even in conjunction with seat positioning switches.

In addition, either or both of the sacral and complementary systems could be pre-set at what was believed to be the most effective position for each to cooperatively, or individually provide the desired support in a particular seat design.

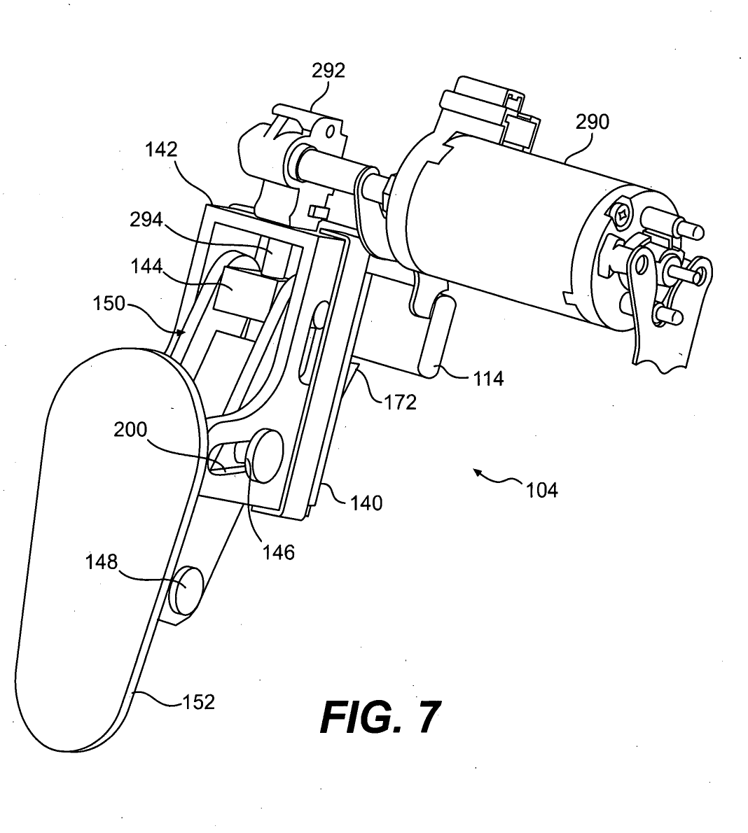

FIG. 7 shows an assembled version of the sacral support assembly 104 in conjunction with motor 290 as a separate or stand alone unit that would not be linked with the upper-back support assembly 102. This sacral support assembly could itself be mounted in a seat and be used as a stand alone device to provide varying amounts of sacra support to a seated used.

Referring again to FIG. 1, sacral support system 104 includes a sacral support member 152, which is located within the seatback at a position that corresponds with the sacrum and sacral-pelvic anatomy of a user. The sacral support member 152 is engineered to support the sacrum and sacral-pelvic anatomy of a user. The sacral support member preferably is formed of a substantially rigid material, such as steel, aluminum, plastics, reinforced plastics or resins, carbon fiber, or combinations of like materials but materials providing a similar level of support can also be used. Sacral support member 152 is generally flat, pear-shaped, and oriented with a larger width dimension positioned at a top portion, and a smaller width located at the bottom portion. This shape and orientation coincides with the shape and orientation of the sacrum of a user. More specifically, an upper portion of sacral support member 152 has a horizontal width of, for example, approximately 3.25 inches. A lower portion of sacral support member 152 has a horizontal width of, for example, approximately 2.6 inches. Preferably, the vertical length of sacral support member 152 is approximately 5.25 inches.

The top width of sacral support member 152 can vary from 3 times the width of the sacrum of a user at the level of the sacral base of the user to approximately equal to the width of the sacrum of a user at the level of the sacral base of the user. The width of sacral support member 152 decreases progressively from a top portion to a bottom portion of sacral support member 152. The width of the bottom portion is approximately greater than or equal to the width of the sacrum of a user at a level corresponding with the bottom portion. However, as discussed below, the dimensions of the sacral support member 152 may vary depending on a variety of factors.

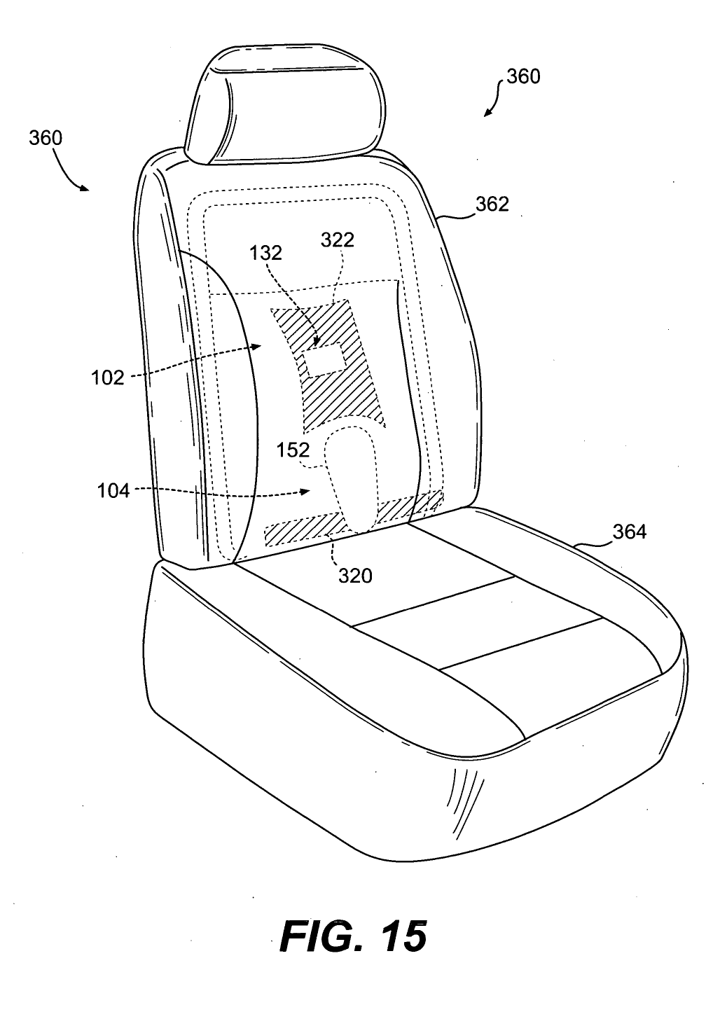

Alternatively, separate load distribution pieces can be provided with a variety of outer shape configurations, including various anatomical designs, they could be thicker or thinner, depending upon cost and/or weight considerations, and can be fabricated from a variety of materials including steel, plastic, aluminum, other metals, combinations of metals and plastic or resin materials, composites and other spring or flexible type materials. Such load distribution mechanisms, devices or materials (collectively herein load distribution material) would be positioned between sacral support member 152 and the sacrum of a user, preferably on the interior of the seat and directly adjacent force transmission plates 132 and 152, and extending outwardly from such plates as illustrated in FIGS. 15-17. For example, as illustrated in FIG. 15, a seat 360 is comprised of a seat back 362 and a seat bottom 364. This seat 360 can have a variety of layers, including, for example, the configuration demonstrated in FIG. 22. As shown in FIG. 15, a load distribution material 320 is positioned in the lower portion of the seatback adjacent the sacral support assembly 104 and the sacral plate 152. This load distribution material 320 might be, for example a thin strip of a light weight metal, such a steel of aluminum, with a thickness varying from about 0.001 to about 0,010 inches, or a plastic or foam strip having a thickness that might vary from about 1/16 inches to about ¼ inches. Such thicknesses can vary depending upon the material being used in the seat, the flexibility or stiffness of the combined layers of material including the foam layer and the seat trim. This load distribution material 320 provides a surface that adjusts to the contours of the sacrum and sacral-pelvic area of a user. Alternatively, the load distribution material 320 can provide a contoured, nesting area for the PSIS's of the ilias. Other preferred arrangements and designs of the load distribution material can include load distribution material having a partially butterfly shape, the use of two or more load distribution materials that work in conjunction with one another and which might be of the same or differing types, or a load distribution material designed to generally cover the anatomical surfaces of the sacral-pelvic area, while also avoiding the potential pressure build-up on the PSIS's of the ilias.

In use, when sacral support member 152 is in an engaged position, the load distribution material 320 improves load distribution across soft tissues of the sacral area of the user and avoids localized pressure on the PSIS's of the ilias.

As is also shown in FIG. 15, seat back 362 can also include an upper-back assembly 102 and in addition to the force transmission plate 132, an extended flexible support or force transmission sheet or plate 322 formed from material as described above for material 320. This extended, flexible plate 322 would spread out the applied force from the upper back assembly 102 so as to extend the effects over a greater portion of the back of a person occupying the seat. Both of these additional force transmission elements 320 and 322, should be flexible and yet strong enough to provide active flexible support extending over a larger area as controlled by the underlying assemblies.

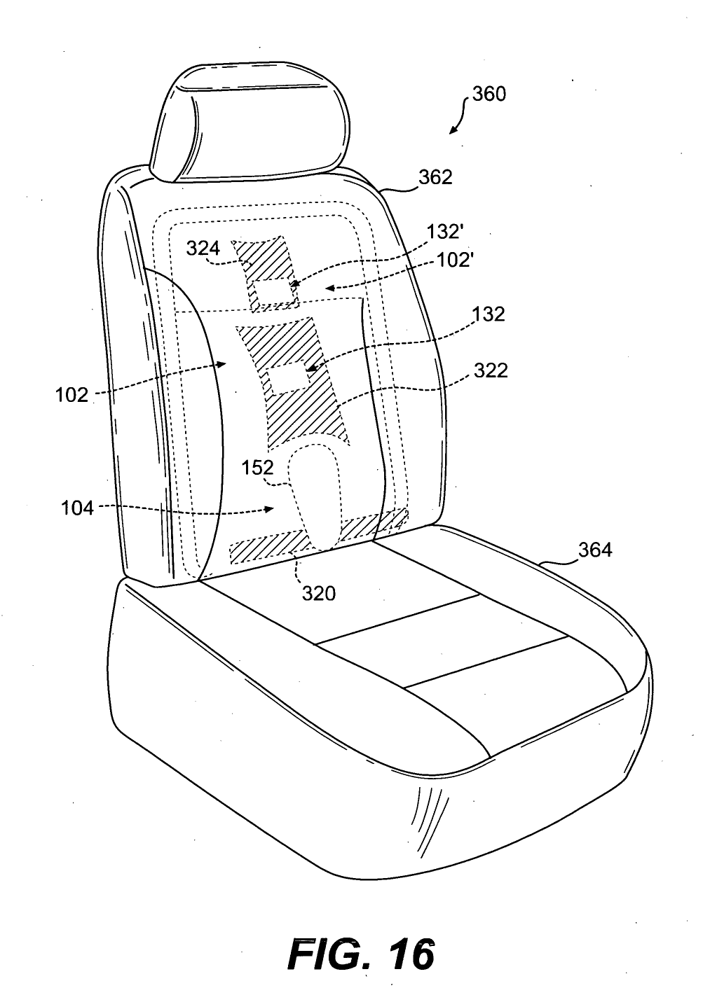

As shown in FIG. 16, an additional upper-back support assembly 102′ could be included as well above the upper-back assembly generally indicated in 102 to provide support for the upper portion of the back, the area specifically between the driver's shoulders. Here too, a load distribution material 324 could be used to spread out the applied force so as to extend the effects over a greater portion between the shoulders. This load distribution material would also be flexible and formed from material such as that described above for material 320.

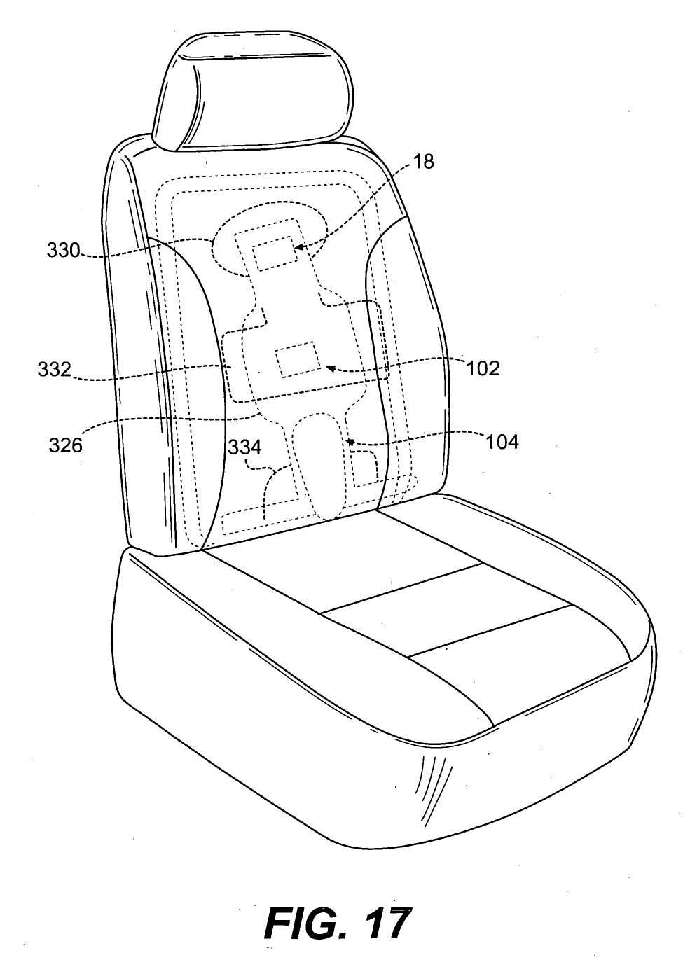

FIG. 17 shows another modified version of seatback 362 as including three support assemblies, 102, 104 and 102′, as well as a one piece load distribution material 326 that spans between the three support assemblies. The shape of load distribution material 326 can vary from the shape shown in FIG. 17, and it is only important that the three support assemblies be linked together thereby. For example, the top portion could have a more circular shape, as shown by dotted line 330, the central portion could have a more horizontally extending section, as shown by dotted line 332, and the lower portion could have a narrower and more upwardly extending shape as shown by dotted line 334.

A variety of support materials may be used as the load distribution material, including compressed foams, plastics, reinforced resins, or strips of lightweight metals, including, for example aluminum. The size and shape of the load distribution material may be altered to accommodate the user's specific anatomical contours and provide improved support and fit. The size and shape of the load distribution can also be altered depending on the particular seat design, including the foam construction and the nature of the trim package. For example, bucket type seats configured for racing applications can be outfitted with relatively rigid load distribution materials, so as to provide greater support and load distribution whereas lightweight seats may be most effective with relatively flexible, lightweight load distribution material.

In addition, cushioning, such as the foam layer in FIG. 22, is preferably provided between the user, the front portion of the seatback and load distribution material. The cushioning may be formed of conventional cushioning materials such as, for example, foam of a variety of densities and thicknesses. Accordingly, in a preferred embodiment, a front portion of the seatback, the trim package, is followed by a cushioning, which is followed by a load distribution material, which is followed by sacral support member 152. Alternatively, a load distribution material can be provided between the trim package in front portion of the seatback and the cushioning layer.

It should be understood that the amount size, shape and flexibility of load distribution material, cushioning, and the dimensions of the sacral support member are related, and can be altered while still achieving the desirable levels of sacral support and contoured fit. For example, to some extent, a sacral support member having smaller dimensions than discussed above may be used if a relatively large load distribution material or cushion is provided between the sacral support member and the user. Conversely, a larger sacral support member than suggested above may be used if relatively less cushioning and load distribution material is provided between the sacral support member and the user. Also, the sacral support member, the load distribution material and cushion may be formed of a unitary structure while still achieving the preferred results of sacral support and contoured fit. Similarly, the load distribution material can be attached directly to the sacral support member. Further, load distribution material may be entirely omitted.

When the sacral support is in an engaged position, sacral support member 152 extends approximately between 1.5 inches and 3 inches forward with respect to a plane created by the seatback. It has been discovered that delivering sacral support member 152 a distance greater than about 3 inches forward of the plane created by the seatback is unnecessary. One embodiment of sacral support system show in FIGS. 1-7 is designed to deliver sacral support member 152 up to 1.25 inches forward of the plane created by the seatback. Notwithstanding this, alternative embodiments of the present invention may deliver sacral support member 152 a maximum distance up to or greater than 3 inches forward of the plane created by a user's back, or a maximum distance less than 3 inches forward of the plane created by a user's back, as described in detail below.

The overall distance of travel of sacral support member 152 toward the user depends on a variety of factors. For example, the overall distance of travel of sacral support member 152 may change depending on the location where the sacral support system is mounted within a seat frame, the size of the seat frame, the type of material used to cover the seat, and the thickness of any cushioning and load distribution material that may be located between the seat cover and sacral support member.

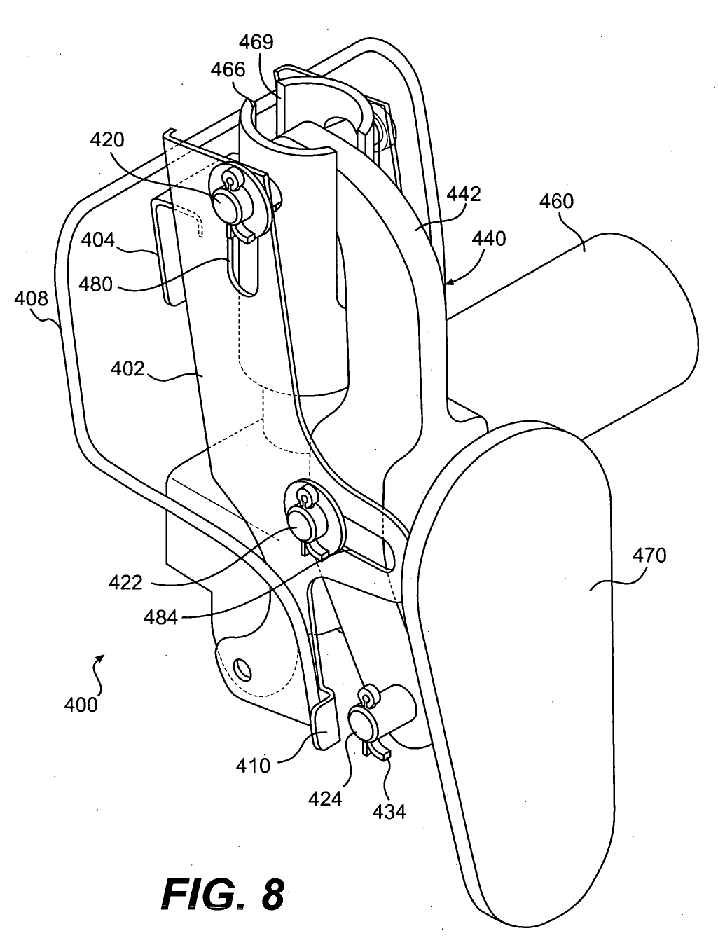

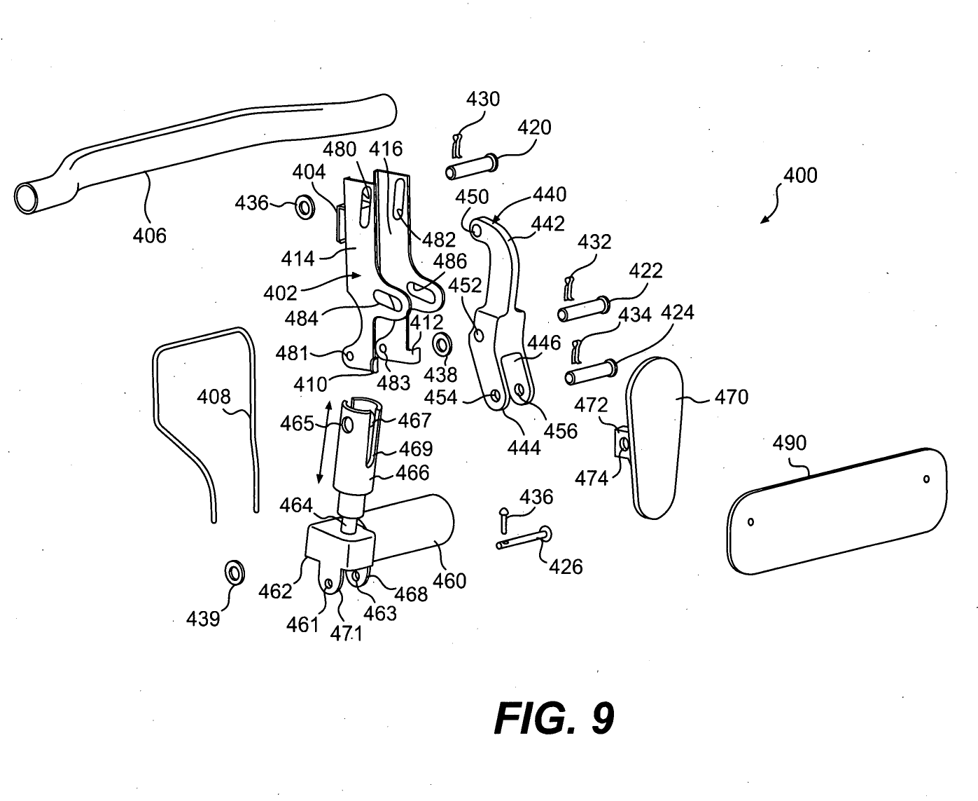

Another embodiment of a sacral support assembly is set forth in FIGS. 8 and 9. This embodiment is a more streamlined version of the sacral support assembly shown in FIGS. 1-7, yet the function is similar, and the sacral plate 470 is still moved in a forward and backward manner relative to the seatback and to the seated user. Here, the sacral support assembly is generally indicated at 400 and is comprised of a main frame 402 having a rear mounting bracket 404 that cooperates with a bar 406 that is part of a seat frame such as that shown in FIG. 1 at 106. Bracket 404 mounts over bar 406 and a spring 408, mounted to frame 402 such as by being welded thereto at tabs 410 and 412, cooperates with the bar 406 to help hold the sacral support assembly in place yet provide some flexibility.

Frame 402 has a U-shaped cross section that is open in the front, with a width of about 43 mm, and has opposing vertically oriented slots 480 and 482 provided in the upper portion of side walls 414 and 416, respectively. Side walls 414 and 416 also include a front wise extending portion including opposing slots 484 and 486, which extend in a some what downwardly sloping angle from vertical. An operating member or lever 440 is comprised of a reverse curved neck portion 442, and a lower portion that includes two downwardly extending, spaced apart leg members 444 and 446. This lever can be about 70 mm in front to back depth, about 35 mm in side to side width and about 150 mm in height. There is also a central section that includes an aperture 452. The operating member 440 also includes an aperture 450 at the top of the neck portion 442 and apertures 454 and 456 located at the bottom of each of leg members 444 and 446, respectively. In addition, apertures 481 and 483 are located at the bottom rear of side walls 414 and 416.

A motor 460 that is controllable as noted previously for motors 270, 280 and/or 290, is mounted via depending tangs 466 and 468 to the bottom of frame 402 by a pin 426 that passes through 481 and 483 in frame 402 and apertures 461 and 463 in tangs 466 and 468. A cotter pin 436 and washer 439 can be used to maintain pin 426 in place.

Pin 420 passes through aperture 450 and also apertures 465 and 467 at the top of a force transmission member 466 that connects to motor 460 by means of an actuating rod 464 that moves vertically by a suitable drive unit 462. The vertical movement of drive member 466 is shown by the double arrow. Drive member 466 is a hollow cylinder which includes an open slot 469 into which the reverse curved neck 442 fits and moves. Motor 460 can be, for example, a Johnson Electric model VD751 series motor, number BC03040.

Pin 422 passes through slots 484 and 486 as well as aperture 452 and cotter pin 432 holds pin 422 in place along with washer 438. Operating member 440 pivots around pin 422 and moves along slots 484 and 486 as the drive member 466 moves vertically under the control of motor 460.

A sacral plate 470 includes a rearwardly extending mounting lug 427 that includes an aperture 474. Sacral plate is about 136 mm high, and has a width, at the widest portion of the upper part thereof, of about 65 mm. Pin 424 passes through apertures 454 ad 456 in legs 444 and 446 as well as aperture 474 to pivotally hold sacral plate 470 to the bottom of operating member 440. Cotter pin 434 holds pin 424 in place as shown in FIG. 8. It might be noted, as well, that sacral plate 152 can be similarly dimensioned.

FIG. 9 also shows an optional load distribution material 490 that can work along with sacral plate 470 to uniformly spread out the forces exerted by sacral plate 470.

In operation, when sacral plate 470 is in its retracted position, similar to that shown for sacral plate 152 in full line in FIG. 6, the drive member 466 will be in its most upward position, pin 420 will be adjacent the top of slots 480 and 482, pin 422 will be located at the rear portion of slots 484 and 486, and legs 444 and 446 will be rotated counter clockwise so that they are predominately located within frame 402. As motor 460 is operated, which can move drive member 466 in a progressive manner downwardly, thus providing an infinite number of positions for sacral plate 470, as drive member 466 moves down within frame 402, pin 420 will slide down slots 480 and 482 and pin 422 will move toward the front of slots 484 and 486. This action rocks neck 422 downward and counter clockwise about pin 422 which simultaneously rotates legs 444 and 446 clockwise as the operating member 440 moves outwardly along slots 484 and 486. This, in turn, moves sacral plate 470 outwardly toward and finally to a fully extended position. The reverse of this operation would pull sacral plate 470 inwardly to a fully retracted position.

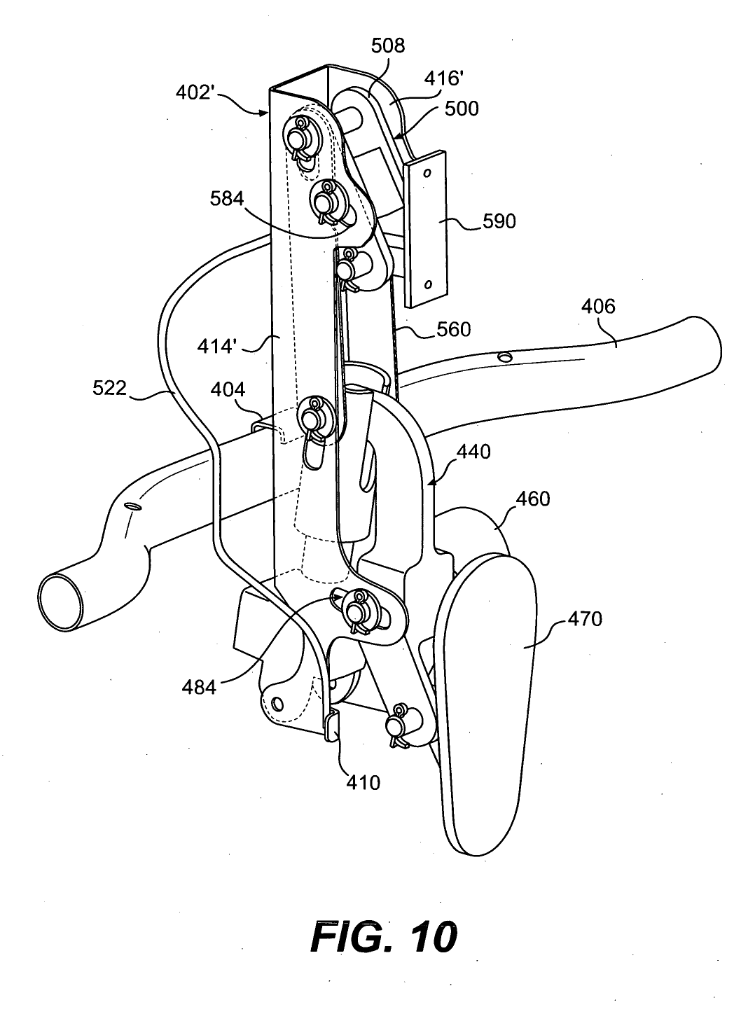

Another embodiment is shown in FIGS. 10-12, and includes as its lower portion many of the elements used in the sacral support assembly shown in FIGS. 8 and 9. Here, however, the embodiment also includes an upper support assembly that is linked to and driven by motor 460 as used previously for the lower sacral support assembly.

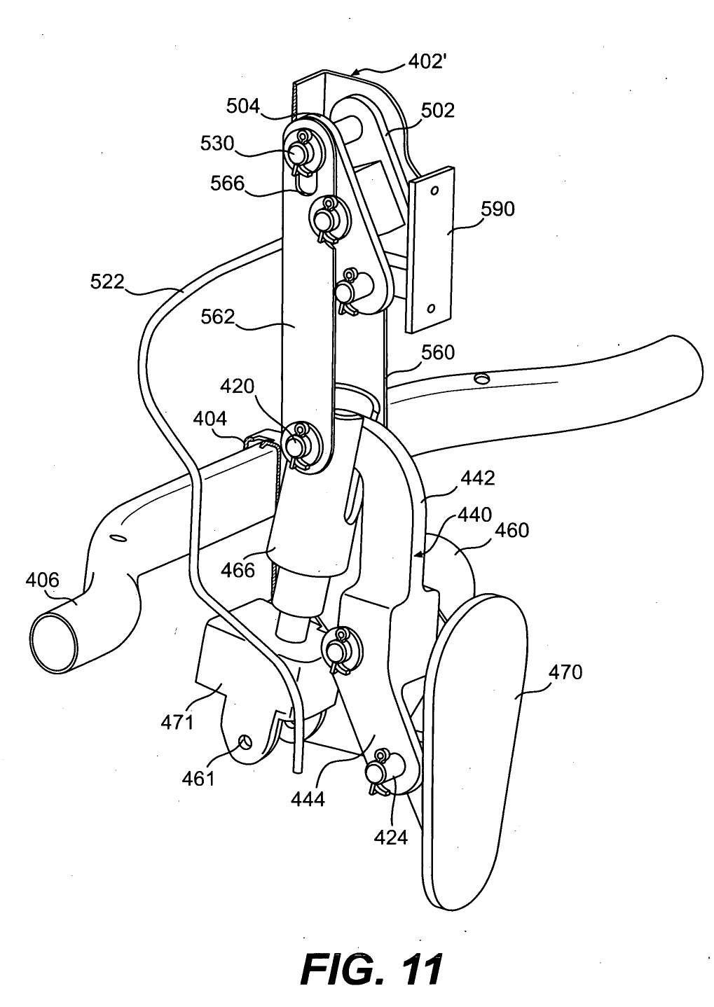

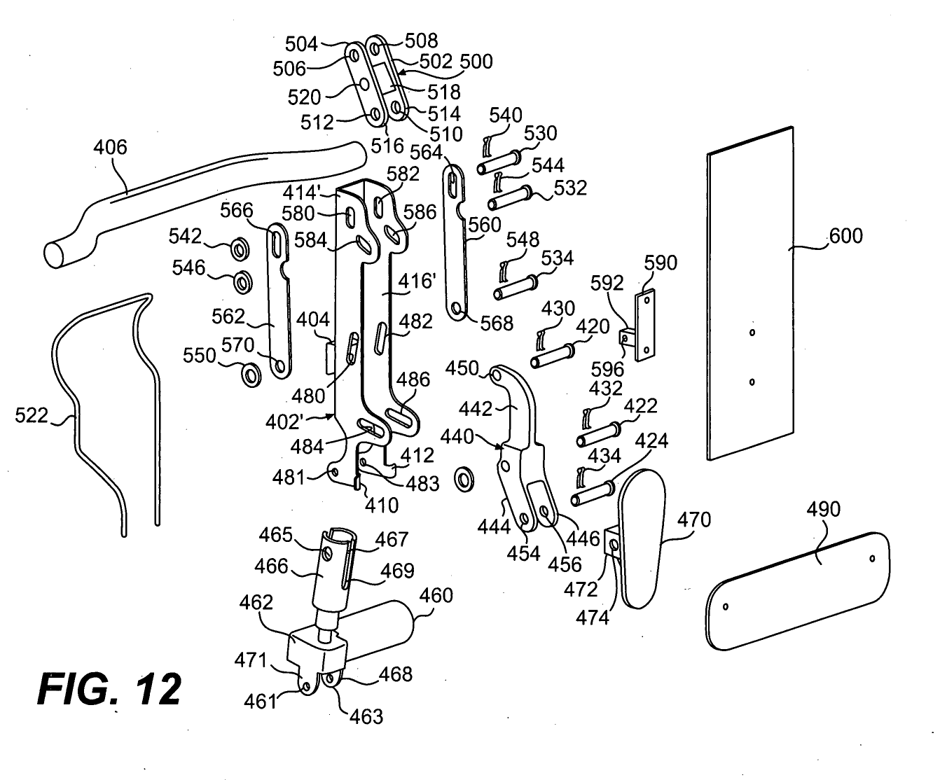

FIG. 10 shows the assembled view while FIG. 11 shows an assembled view from which portions have been cut away for better clarity, and FIG. 12 shows an exploded view of each of the elements used.

The frame used in this embodiment is vertically longer and is identified as 402′. It has a height of about 289 mm and a similar width to the FIG. 9 embodiment of about 43 mm. Frame 402′ includes an extended upper portion with side walls 414′ and 416′ further including upper slots 580- and 582 as well as downwardly sloping slots 584 and 586.

An H-shaped member 500 is used and is comprised of side pieces 502 and 504, a pair of upper opposed apertures 506 and 508, and a pair of opposed lower apertures 510 and 512. A central piece 518 joins the two sides 502 and 504, and forms them into a one piece unit, for example, by being welded together. The central section 518 also includes an aperture 520.

A pair of elongated side mounted drive members, 560 and 562, are connected to drive member 466 by pin 420. These drive members include an upper slot 564 and 566, as well as a lower aperture 568 and 570. With reference to FIGS. 10-12, the H-shaped member is located within frame 402′ by pin 530 that passes through slot 564, apertures 508 and 506, slot 580 and slot 566. Pin will be held in place by cotter pin 540 and washed 542. The central aperture 520 in H-shaped piece is connected to slots 584 and 586 by pin 532, which is held in place by cotter pin 544 and washed 546. An upper force transmission plate 590, which includes a rear tang 592 and aperture 596, is pivotally mounted to the H-shaped member by pin 534 which passed through apertures 510 and 512 and 596. Pin 534 is held in place by cotter pin 548. In addition, a load distribution material 600 is also shown as being available for use with the upper force transmission plate in order to provide a mechanism to extend the effective area of the upper support assembly.

Spring 522 is longer, but this assembly is held on frame member 406 by a rear bracket 404 and spring 522 will contact frame 406 as shown in FIGS. 10-11.

In operation, motor 460 will control the vertical movement of drive member 466. With drive member 466 in its up or extended position, both sacral plate 470 and the upper back or lumbar plate 590 will be in their retracted positions. In that position, lumbar plate 590 can be flush with, or aligned with the front face of the sacral plate 470, or alternatively, plate 590 can be off set rearwardly a slight amount from the front face of the sacral plate 470, for example by about 12 mm. This aligned or offset relationship will vary depending upon seat design, the slope in angle of the seat and/or internal seat configurations. As drive member 466 starts to move downwardly, both the operating member 440 and the H-shaped member 500 will begin to move. Pin 420 connects drive member 466 to the hooked end 442 of the operating member 440 and to the drive members 560 and 562 which in turn connect to the top portion of H-shaped member 500. H-shaped member 500 will be pulled downwardly and the central portion, together with pin 532 will begin to slide outwardly causing the H-shaped member 500 to pivot its bottom portion outwardly which moves the lumbar force plate 590 outwardly. The operation of the lower operating member 440 will be the same as described above.

FIG. 13 shows an additional embodiment that is in most respects the same as the embodiment shown and described in FIGS. 10-12 except that here the actuation is not under the control of a motor, but rather is a manually activated system. The frame and operating elements are the same as in the FIG. 10-12 embodiment, but the motor 460 is replaced by a manual control system 700 that includes a control wire or cable 702 with a cable connection 710 at one end and an actuation knob 712 at the opposite end, and a cable holder 704 that is mounted to the bottom of frame 402′ by a screw 706 and a nut 708, with screw 706 passing through apertures 481 and also 483 (not shown). Cable holder 704 will receive cable 702 and permit the cable end connection 710 to be connected to pin 422 so that the cable will be able to pull and push the drive members 560 and 562 as well as the operating member 440, by movement of the upper end of the curved end of portion 442. The movement of cable end 710 will be under the control of the actuation knob 712 as controlled by a seat user, and could be either a push-pull or rotation control mechanism. It is preferred that the actuation knob 712 be located adjacent the side of the seat, and within reach of a user, but it may be positioned at other locations as well.

The parts of the embodiments disclosed herein are preferably constructed from light weight metal, including aluminum, or steel, but molded plastic parts, or reinforced resins, such as, for example, ZYTEL® (70G33HS1L-NC010-33% glass reinforced nylon resin), or a PPO resin like General Electric's Noryl PPO resin, for example GTX-910 could be used as well. It is important that the support assemblies be light weight yet sufficiently strong to operate correctly and effectively for the life of the seat. Thus, the parts should be fabricated from materials that will provide the functions and results while not be overly strong, heavy or bulky. The main support bracket, for example 402′ in FIG. 12, might be made from a metal, while the remaining parts could be made from a resin material. Alternatively, all parts could be made from metal or all parts could be made from resin material. This, it is contemplated that a combination of resin and metal parts might be used as well as all one material.

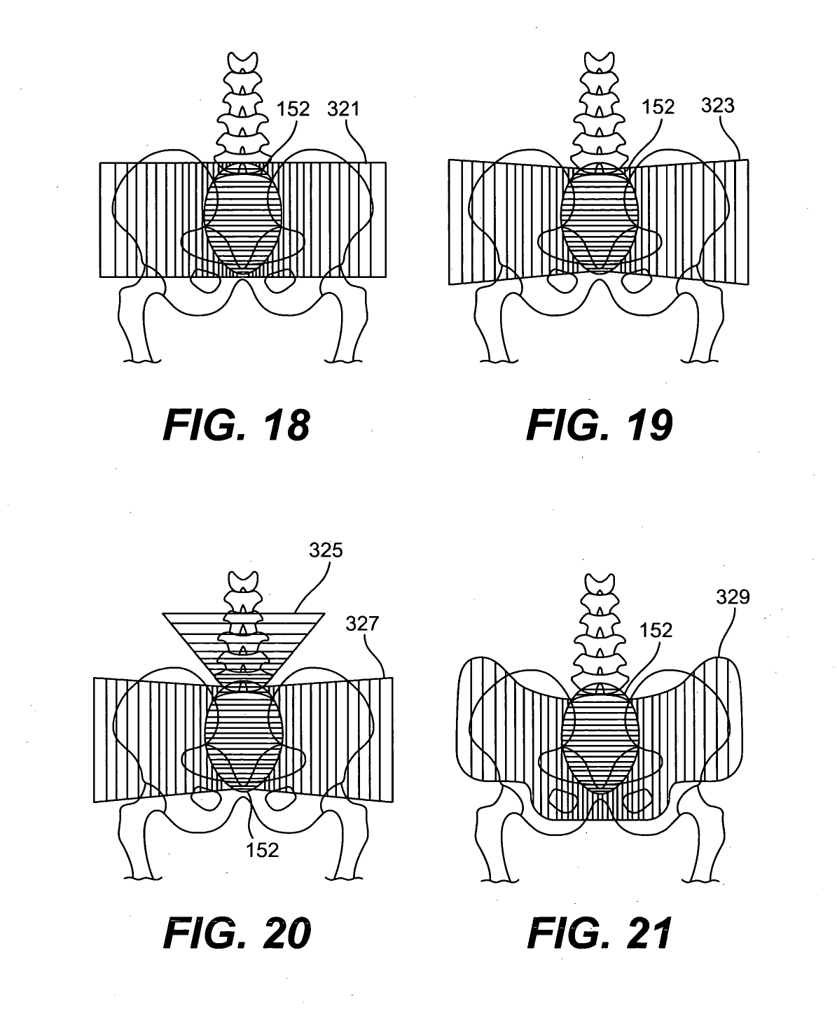

FIGS. 18-21 show varying forms of load distribution materials. FIG. 18 illustrates use of a load distribution material 321 in the form of a rectangular plate which provides a contoured, nesting area for the PSIS's of the ilias. Other preferred arrangements and designs of the load distribution material can be contemplated as well and several other forms are depicted in FIGS. 19-21. For example, FIG. 19 illustrates an embodiment in which the load distribution material 323 exhibits a partially butter flied shape. FIG. 20 illustrates an embodiment in which two load distribution materials 325 and 327 are provided. FIG. 21 illustrates a load distribution material 329 that has been designed to generally cover the anatomical surfaces of the sacral-pelvic area, while also avoiding the PSIS's of the ilias.

In use, when sacral support member 152 and/or 470 is in an engaged position, the load distribution material improves load distribution across soft tissues of the sacral area of the user and avoids localized pressure on the PSIS's of the ilias. Preferably, conventional support materials may be used to provide load distribution, such as compressed foams, plastics or strips of lightweight metals, for example aluminum. The size and shape of the load distribution material may be altered to accommodate the user's specific anatomical contours and provide improved support and fit. The size and shape of the load distribution can also be altered depending on the particular seat trim package. For example, bucket type seats configured for racing applications can be outfitted with relatively rigid load distribution materials, so as to provide greater support and load distribution.

In addition, cushioning is preferably provided between the user, the front portion of the seatback and load distribution material. The cushioning may be formed of conventional cushioning materials such as foam. Accordingly, in a preferred embodiment, a front portion of the seatback is followed by a cushioning, which is followed by a load distribution material, which is followed by sacral support member 152 and/or 470. Alternatively, a load distribution material can be provided between the front portion of the seatback and the cushioning.

It should be understood that the amount of load distribution material, cushioning, and the dimensions of the sacral support member are related, and can be altered while still achieving the desirable levels of sacral support and contoured fit. For example, to some extent, a sacral support member having smaller dimensions than discussed above may be used if a relatively large load distribution material or cushion is provided between the sacral support member and the user. Conversely, a larger sacral support member than suggested above may be used if relatively less cushioning and load distribution material is provided between the sacral support member and the user. Also, the sacral support member, the load distribution material and cushion may be formed of a unitary structure while still achieving the preferred results of sacral support and contoured fit. Similarly, the load distribution material can be attached directly to the sacral support member.

The foregoing description has been directed to specific embodiments of the present invention. It will be apparent to those with ordinary skill in the art that modifications may be made to the described embodiments of the present invention, with the attainment of all or some of the advantages. For example, the techniques of the present invention may be utilized for seats other than in automobiles or trucks, including seats for airplanes, wheel chairs, office furniture, home furniture and the like. Therefore, the object of the appended claims is to cover all such variations and modifications as come within the spirit and scope of the invention.

I claim:

1. A device adapted to be mounted within a seat for progressively providing varying amounts of force to a seat user's sacral anatomy comprising:
a frame;
an actuating mechanism operably connected to the frame; and
a sacral support member operatively connected to the actuating mechanism that is moved through a range of motion relative to the frame including a combination of vertical and horizontal movements, to thereby move the sacral support member relative to the user's sacral anatomy.

2. The device of claim 1, wherein the sacral support member has a top portion with a greater horizontal width dimension than a bottom width dimension.

3. The device of claim 1, wherein the sacral support member has a top portion with a horizontal width dimension of about 3.25 inches, and a bottom portion has a width dimension of about 2.6 inches.

4. The device of claim 1 further comprising:
a second actuating mechanism operably connected to the frame; and
a complementary support member, operably connected to the second actuating mechanism and spaced from the sacral support member.

5. The device of claim 4, wherein the complementary support member is configured to support one of the thoracic region of the spine of a user, the thoracic-lumbar region of the spine of a user, the lumbar region of the spine of a user, and the lumbar-sacral region of a user.

6. The device of claim 1, wherein the actuating mechanism comprises a drive mechanism, and an articulable member having a first portion movably connected to the frame, a second portion movably attached to the drive mechanism and a third portion movably attached to the sacral support member, so that as the sacral support member is moved through the range of motion the articulable member moves through a range of motion exhibiting a combination of both vertical and horizontal components.

7. A force transmitting device comprising: a frame, a drive member, movably mounted to the frame, said drive member having a first end operatively connected to a drive motor fixed to the frame and a second end pivotally connected to a force plate, the drive motor being adapted to move the first end between extended and retracted positions relative to the frame causing drive member to rotate and causing the second end to move the force plate relative to the frame between its own extended and retracted positions.

8. The force transmitting device as in claim 7 further including an enlarged frame having an upper portion, a second drive member operatively positioned within the upper portion so as to be movable relative thereto, an extension interconnecting the drive motor and the second drive member to actuate the second drive member between extended and retracted positions, and a second force plate movably attached to the second drive member which will move between extended and retracted positions relative to the upper portion of the frame and the seat.

9. The force transmitting device as in claim 8 further including a seat frame and a clamp assembly securing the frame to the seat frame.

10. The force transmitting device as in claim 8 further including a seat frame and a spring member positioned between the frame and the seat frame to provide a resistance force there between.
